(19) United States
(12) Patent Application Publication
(10) Pub. No.: US 2011/0319790 A1
(43) Pub. Date: Dec. 29, 2011

(54) PHARMACEUTICAL COMPOSITION AND SYSTEM FOR PERMEABILIZING FETAL MEMBRANES

(75) Inventors: Joseph Kost, Omer (IL); Mordechai Halak, Raanana (IL); Drora Shmilovitch, Meitar (IL); Tamar Triatel, Beer-Sheva (IL); Rivka Goldbart, Lehavim (IL); Aharon Azaguri, Kiryat Gat (IL); Lior Wollach, Kiryat Motzkin (IL)

(73) Assignee: B.G. NEGEV TECHNOLOGIES AND APPLICATIONS LTD., Beer Seva (IL)

(21) Appl. No.: 13/130,916

(22) PCT Filed: Nov. 25, 2009
(86) PCT No.: PCT/IL2009/001113

§ 371 Date: Sep. 14, 2011

Related U.S. Application Data
(60) Provisional application No. 61/193,407, filed on Nov. 25, 2008.

(51) Int. Cl. A61M 37/00; A61K 31/185; A61K 31/201; A61K 31/20; A61B 5/00; A61K 31/047; A61K 31/14; A61K 31/4015; A61K 31/015; A61P 43/00; A61K 31/445; A61K 31/167

(52) U.S. Cl. 600/582; 514/330; 514/578; 514/560; 514/558; 514/626; 514/738; 514/642; 514/424; 514/763; 604/22

(57) ABSTRACT

Provided is a pharmaceutical composition for permeabilizing fetal membranes including an active ingredient having a log K in the range of 2 to 4, where K is the octanol/water partition coefficient. The active ingredient may be, for example, bupivacaine, sodium lauryl sulfate or oleic acid. Further provided is a system for transfetal membrane transport. The system includes a probe unit adapted for insertion into a female reproductive tract and releasing a substance onto fetal membranes that permeabilizes the membranes. The system is also configured to apply ultrasound radiation to the fetal membranes to further increase the membrane permeability.

### FIELD OF THE INVENTION

This invention relates to pharmaceutical compositions and to medical devices, and more particularly to gynecological compositions and medical devices.

### BACKGROUND OF THE INVENTION

The following prior art publications are considered relevant for an understanding of the invention.
1. Karande P., Jain A. & Mitragotry S., Discovery of transdermal penetration enhancers by high-throughput screening, J Nature Biotechnology, 22, 2 (2004).
2. Suhonen M., Bouwstra J., & Urtti A., Chemical enhancers of percutaneous absorption in relation to stratum corneum structural alteration. J Control Release 59, 146-161 (1999).
3. Mitragotri S., Effect of bilayer disruption on transdermal transport of low molecular weight hydrophobic solutes. Pharm. Res. 18, 1022-1028 (2001).
4. Williams A C, Barry B W. Chemical permeation enhancement, in: Elka Touitou, Brian W. Barry (Ed.), Enhancement in Drug Delivery, CRC Press, 233-254 (2006).
5. U.S. Pat. No. 6,773,418 to Sharrow et al.
6. U.S. Pat. No. 6,264,638 to Contente.
7. U.S. Pat. No. 6,139,538 to Houghton et al.
8. U.S. Pat. No. 5,988,169 to Anderson et al.
9. U.S. Pat. No. 7,425,340 to Grenier et al.

Prenatal testing involves testing a fetus for the presence of various hereditary or spontaneous genetic disorders, such as Down syndrome. One of the most common procedures for detecting abnormalities before birth is amniocentesis in which a sample of the fluid surrounding the fetus (amniotic fluid) is obtained. In amniocentesis, after anesthetizing an area of abdominal skin, a needle is inserted through the abdominal wall into the amniotic cavity. During the procedure, ultrasonography is performed so that the position of the fetus can be monitored and the needle guided into place without touching the fetus. Amniotic fluid is aspirated through the needle into a syringe, and the needle is then removed. Another method of fetal examination is chorionic villus sampling (CVS). Both amniocentesis and CVS are invasive, and as such carry a small but definite risk to the mother and fetus. After amniocentesis, the chance of miscarriage due to the procedure is about 1-2 in 100. Also these invasive tests for evaluating fetus health can be preformed only at relatively late stages of the pregnancy (from week 13) and two more weeks are required to obtain the results. Moreover, many women fear of these invasive tests because of the pain and risk of miscarriage.

In recent years, numerous efforts have been made to find alternatives to invasive procedures. One of these methods involves applying a chemical penetrating enhancer (CPE) onto a biological membrane in order to enhance the permeability of the membrane. CPEs alter the partition coefficient of substances to be transported across the membrane, either by modification of the substances to be transported or altering the membrane structure. In transdermal applications, CPEs usually enhance skin permeability by altering the stratum corneum structure [1-2]. Since the stratum corneum consists of dead keratinized cells, disruption of its structure does not induce irritation [3].

CPEs can be divided into major classes based on their chemical structure. The major classes of CPEs are: water, sulfoxides, azone, pyrrolidones, fatty acids, alcohols and glycols, surfactants, urea, essential oils, terpenes and terpenoids, phospholipids, and ceramide analogs [4].

Ultrasound has been used in a number of medical applications. Examples of clinical applications of ultrasound include imaging, stimulation of the healing of soft tissue, during topical application of a medication, and for enhancement of transdermal drug delivery into the circulatory system. In addition, ultrasound has also been used for selectively altering the permeability of cell membranes. This alteration is reversible and the effect can be controlled as to its extent and rate.

U.S. Pat. No. 6,773,418 to Sharrow et al discloses a device for delivering an agent to the uterine cervix. The device includes a chamber that is engaged with the cervix. An agent delivery port in fluid communication with the chamber is provided for delivery of the agent to the uterine cervix. A vacuum port in fluid communication with the chamber allows application and retention of vacuum pressure to the chamber to seal the chamber to the cervix and prevent leakage of the agent away from the cervix.

U.S. Pat. No. 6,264,638 to Contente discloses a device for introducing agents, including drugs and other substances, into the vaginal canal. The device may also be used to collect discharges from the canal. The device has an elastic rim surrounding a flexible film and is lodged in the vaginal canal.

U.S. Pat. No. 6,139,538 to Houghton et al discloses an apparatus for iontophoretically delivering an agent to a uterus or cervix. The apparatus comprises a probe sized to fit within the cervical canal. The device includes a reservoir for containing the agent to be delivered and a pair of electrodes for iontophoretically delivering the agent into the uterus or cervix.

U.S. Pat. No. 5,988,169 to Anderson et al discloses a vaginal insert for delivering an agent into a female urogenital tract. The insert has first and second portions projecting outward from a main portion where at least one of the projecting ends has means for containing the agent. The projecting ends of the first and second portions are configured to engage the anterior vaginal wall while the main portion engages the posterior vaginal wall, thereby positioning the projecting end of the first portion proximal to one side of the urogenital tract and positioning the projecting end of the second portion proximal to an opposite side of the urogenital tract.

U.S. Pat. No. 7,425,340 to Grenier et al discloses a composition for transdermal transmucosal administration comprising a therapeutically effective amount of an anticholinergic or antispasmodic agent, and a urea-containing compound in an amount sufficient for enhancing permeation of the anticholinergic agent, and a carrier system suitable for topical or transdermal drug delivery. The composition may be used for treating urinary incontinence. The composition may be administered via buccal and sublingual tablets, suppositories, vaginal dosage forms, or other passive or active transdermal devices for absorption through the skin or mucosal surface.

### SUMMARY OF THE INVENTION

In its first aspect, the present invention provides a pharmaceutical composition for permeabilizing fetal membranes. The pharmaceutical composition of the invention comprises an active ingredient and a physiologically acceptable carrier wherein the active ingredient comprises any one or more ingredients having a log K in the range of 2-4, where K is the octanol/water partition coefficient of the active ingredient. The inventors have found that substances having a log K in the range of 2 to 4 are capable of permeabilizing fetal membranes.

For example, the composition may contain bupivacaine in a concentration from 0.1% to 1% (wt/vol), or sodium lauryl sulfate (SLS) in a concentration from 0.1% to 10%, or oleic acid in a concentration from 0.1% up to 2%. Alternatively, the pharmaceutical composition may comprise a mixture of bupivacaine, SLS, and limonene. In this case, the bupivacaine may have a concentration from 0.1% to 10%, the limonene may have a concentration from 1% to 10%, and the SLS may have a concentration from about 0.1% to 10%.

The pharmaceutical composition of the invention may be in the form of a liquid or a paste in order to allow it to be applied to the amniotic membrane.

In its second aspect, the invention provides a system for permeabilizing a fetal membrane. The system of the invention comprises a probe unit adapted for insertion through a vagina into a female reproductive tract. The probe unit has a reservoir and a slender shaft. A delivery system is configured to release a substance stored in the reservoir from the distal end of the shaft. The system further comprises an ultrasound source located at the distal end of the shaft, and a control unit configured to activate the ultrasound source. The system is used to release a pharmaceutical composition of the invention from the reservoir and apply the composition to the fetal membrane to be permeabilized. Activation of the ultrasound enhances the permeabilization caused by the active ingredient in a synergistic manner.

The shaft may be curved or bent to form a vaginal portion and a cervical portion in order to facilitate insertion into the female reproductive tract and delivery of the distal end of the shaft to the cervix. Alternatively, the shaft may be flexible.

The system of the invention may further comprise a collecting system configured to collect substances around the distal end of the shaft. This may be used to collect substances released from the amniotic sac after permeabilization in accordance with the invention. The collection system may comprise a vacuum system or a solution of high osmotic pressure that collects fluids by osmosis or vacuum

The invention also provides a method for permeabilizing a fetal membrane comprising applying to the fetal membrane a pharmaceutical composition of the invention. The pharmaceutical composition may be applied to the fetal membrane by releasing the pharmaceutical composition from the distal end of the shaft of a system of the invention. The method may further comprise applying ultrasound radiation to the fetal membrane. The ultrasound radiation may have a frequency in the range of 20 kHz to 100 kHz.

Further provided by the invention is a method for delivering one or more substances into an amniotic sac comprising permeabilizing fetal membranes of the amniotic sac by the method of the invention and applying the one or more substances to the permeabilized membrane. The one or more substances may be applied (with or without ultrasound) to the membrane by releasing the one or more substances from the distal end of the shaft of a system of the invention.

The invention further provides a method for collecting a fluid from an amniotic sac comprising permeabilizing fetal membranes of the amniotic sac by the method of the invention and collecting fluid released from the amniotic sac. The fluid released from the amniotic sac may be collected into the reservoir of a system of the invention or detected in situ by a sensor at the distal end of the shaft.

Thus, in its first aspect, the present invention provides a pharmaceutical composition for permeabilizing fetal membranes comprising one or more active ingredients and a physiologically acceptable carrier wherein the one or more active ingredients have a log K in the range of 2 to 4, where K is the octanol/water partition coefficient.

In its second aspect, the invention provides a system for transfetal membrane transport comprising:
(a) a probe unit adapted for insertion through a vagina to a into a female reproductive tract comprising a shaft having a proximal end and a distal end;
(b) an ultrasound source located at the distal end of the shaft;
(c) a reservoir;
(d) a delivery system configured to release a substance in the reservoir from the distal end of the shaft; and
(e) a control unit configured to activate the ultrasound source.

In another of its aspects, the invention provides a method for permeabilizing a fetal membrane comprising applying to the fetal membrane a pharmaceutical composition of the invention.

In still another of its aspects, the invention provides a method for delivering one or more substances into an amniotic sac comprising permeabilizing fetal membranes of the amniotic sac by the method of the invention and applying the one or more substances to the permeabilized membrane.

In yet another aspect, the invention provides a method for collecting a fluid from an amniotic sac comprising permeabilizing fetal membranes of the amniotic sac by the method of the invention and collecting fluid released from the amniotic sac.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows a system for trans-fetal membranes transport according to one embodiment of the invention;

FIG. 2 shows a probe unit for use in the embodiment of FIG. 1;

FIGS. 3a and 3b show the probe of FIG. 2 inserted in a vagina and a cervix;

FIG. 4 shows a system for trans-fetal membranes transport according to another embodiment of the invention;

FIG. 5 shows a probe unit for use in the embodiment of FIG. 1;

FIGS. 6a and 6b show the probe of FIG. 2 inserted in a vagina and a cervix;

FIG. 7 shows a system for in vitro trans-fetal membranes transport;

FIG. 8 shows the effect of different CPEs on postpartum human fetal membranes permeability; and

FIG. 9 shows a synergistic effect on the permeability of fetal membranes of a substance having a log K in the range of 2 to 4 and ultrasound radiation.

### DETAILED DESCRIPTION OF EXEMPLARY EMBODIMENTS

### Permeabilization of Amniotic Membranes

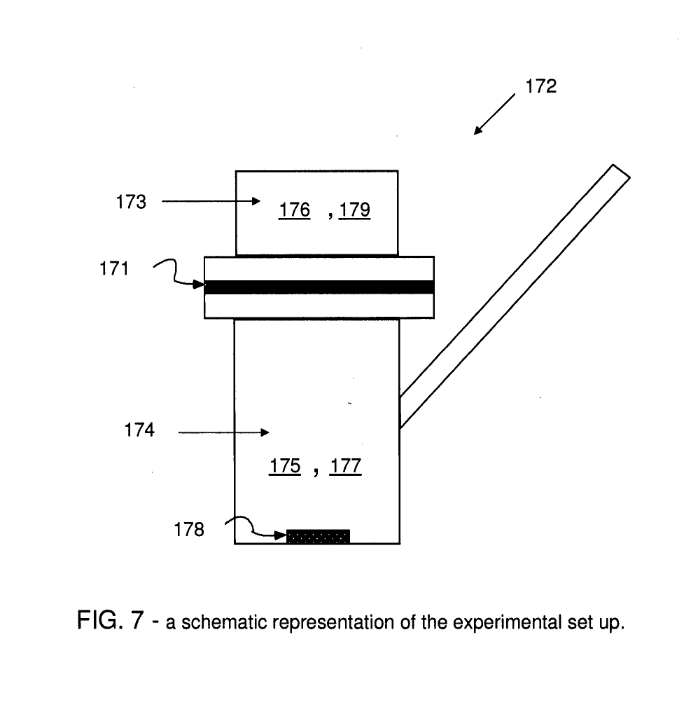

The permeability of postpartum human fetal membranes (obtained from Hillel Yaffe Medical Center, Israel) upon exposure to different substances was determined. The experimental set-up used is shown schematically in FIG. 7. As shown in FIG. 7, a fetal membrane 171 (a piece of a gestational sac) was mounted on a vertical glass diffusion cell 172. The membrane 171 was first incubated with a substance 176 added to the donor compartment 173. After 30 minutes of incubation, the substance was removed from the donor compartment 173. The donor compartment was then washed with PBS, and the donor and receiver compartments were filled with 0.01M phosphate buffered Saline (PBS) 175. 5 mL of a 0.5 mg/mL Dextran solution in PBS (average molecular weight of the Dextran 77 KDa) conjugated to the fluorescent label FITC (fluorescein isothiocyanate-Dextran)) 179 was added to the donor compartment 173. The diffusion cell 172 was protected from light in order to prevent fluorescent bleaching of the FITC. 1 mL samples were withdrawn from the receiver compartment 174 at various times with constant stirring of the receiver compartment, and the concentration of the FITC in the samples was determined spectofluorimetrically.

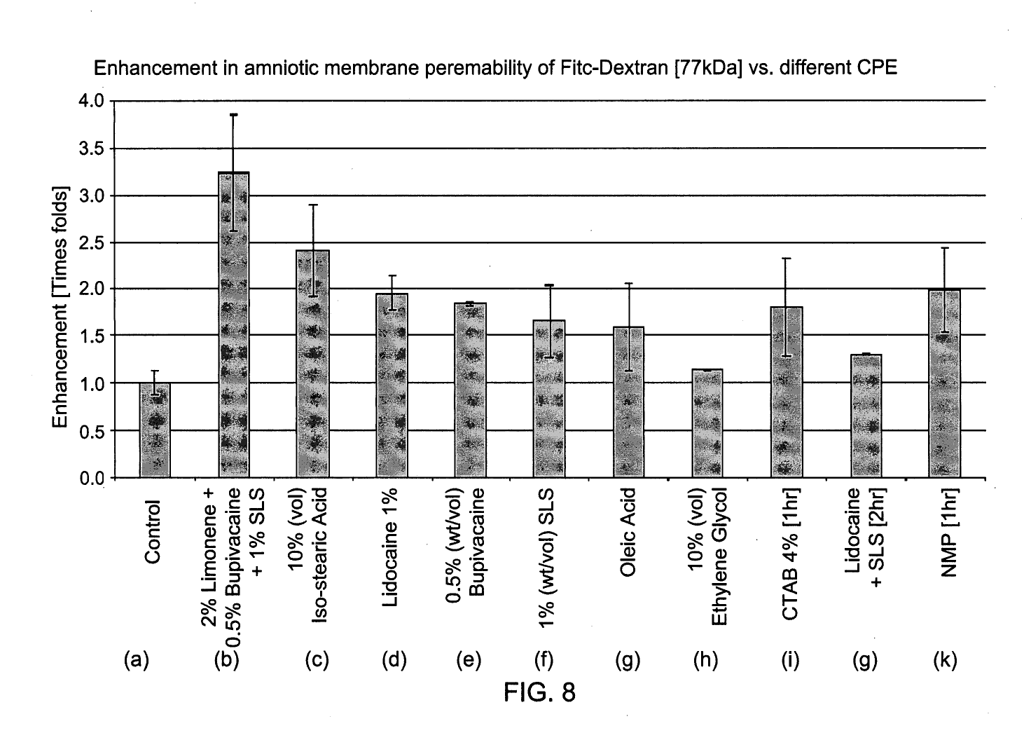

From the rate of increase in the concentration of the fluorescence in the receiver compartment 174, the extent of permeability enhancement of the membrane, in comparison to a control experiment was calculated. FIG. 8 shows the enhancement in permeability of the membrane treated by several substances having a log K in the range of 2 to 4 compared to a control in which the incubation step was omitted (column a in FIG. 8). The results shown in FIG. 8 were obtained with the maternal side facing the donor compartment. Of the substances tested, a maximal effect was observed with a mixture of 1% sodium lauryl sulfate (SLS), 2% limonene, and 0.5% bupivacaine (b). Enhanced permeability was also observed with 10% iso-stearic acid (c), 1% lidocaine (d), 0.5% bupivacaine (e), 1% SLS (f), 1.5% Oleic acid (g), 10% ethylene glycol (h), 4% Cetyl trimethylammonium bromide (CTAB) (i), a mixture of 1% lidocaine and 1% SLS (j), and N-methy-2-pyrrolidone (NMP) (k).

Results similar to those presented in FIG. 8 were obtained with the maternal side facing the receiving compartment (not shown).

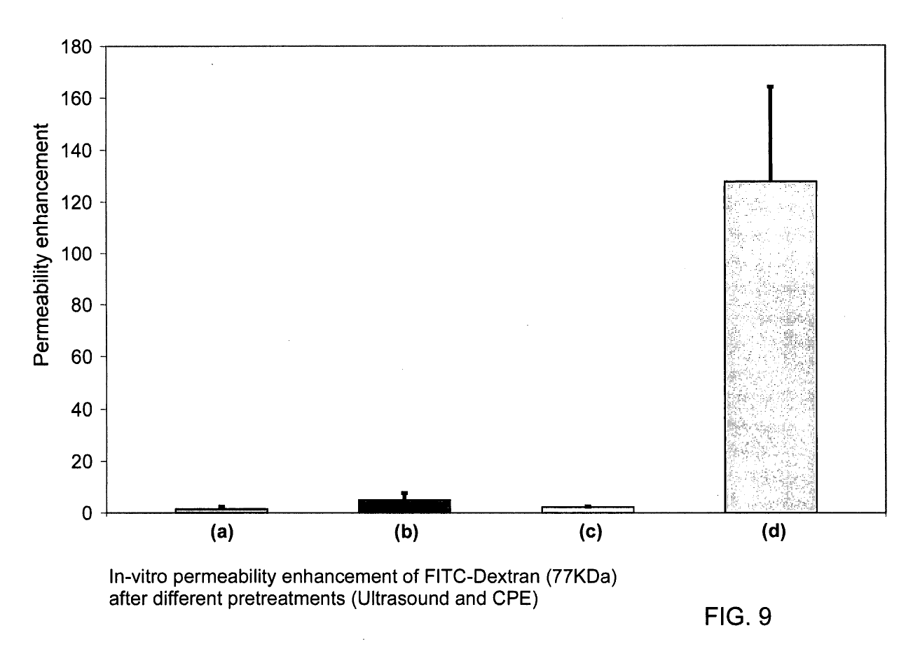

FIG. 9 shows enhancement of permeability of fetal membranes after incubation with the above mentioned mixture comprising a combination of 1% Sodium Lauryl Sulfate (SLS), 2% limonene, and 0.5% bupivacaine. a substance having a log K in the range of 2 to 4 (b), after exposure to ultrasound radiation (c), and after exposure to both the mixture and ultrasound (c), in comparison with control membranes which were not exposed to the mixture or to ultrasound radiation (a). Exposure to the substance and the ultrasound radiation had a synergistic effect on the permeability.

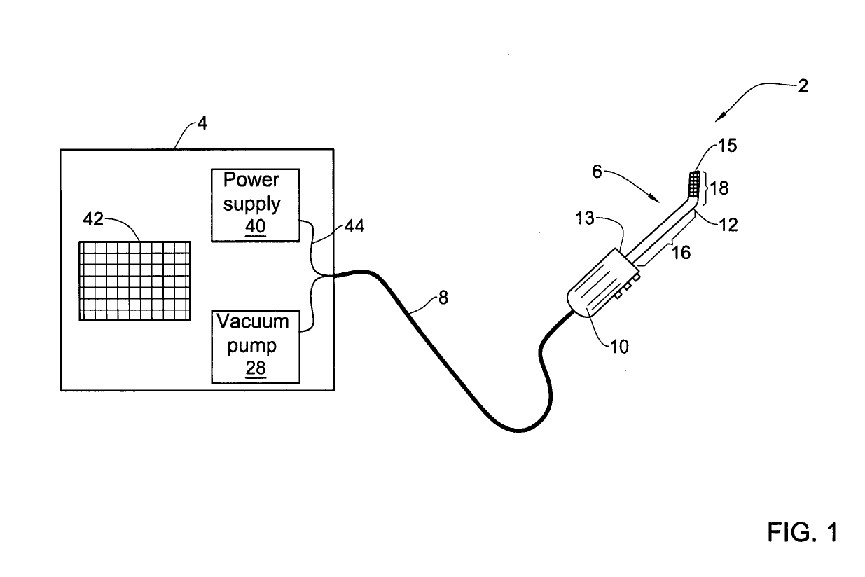

FIG. 1 shows a system generally indicated by 2 for transfetal membrane transport in accordance with one embodiment of the invention. As explained below, the system 2 may be used to generate transport through the fetal membranes from the interior of the gestational sac to the exterior, for example, to obtain a sample of amniotic fluid or coelomic fluid. Alternatively, the system 2 may be used to generate transport through the fetal membranes into the gestational sac, for example, to deliver a drug into the sac.

The system 2 comprises a control unit 4 and a probe unit 6 which is attached to the control unit 4 via a harness 8. The probe unit 6 has a handle 10, a shaft 12, a proximal end 13 and a distal end 15. The shaft 12 may be rigid and permanently bent or curved to form a vaginal portion 16 and a cervical portion 18. Alternatively, the shaft may be flexible so as to be bendable into an angled shape having a vaginal portion and a cervical portion to match the anatomy of the recipient of the treatment.

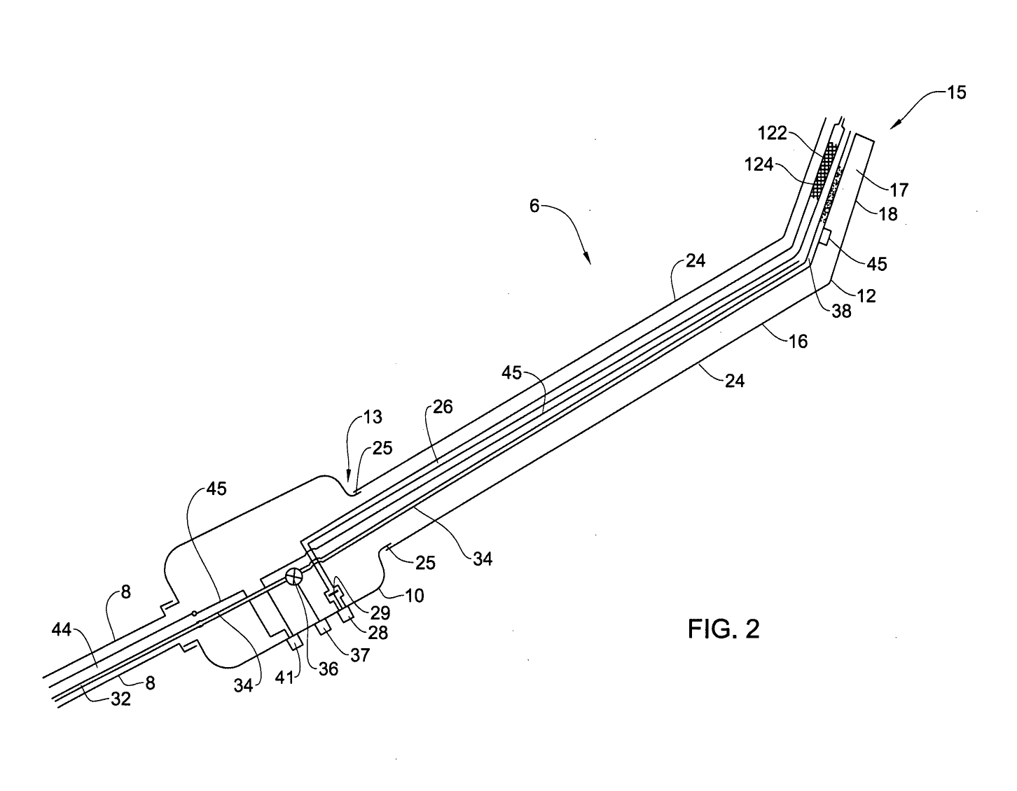

FIG. 2 shows the probe unit 6 in greater detail. The cervical portion 18 comprises an ultrasound source 17 that emits ultrasound waves from the distal end 15 of the probe unit 6. The ultrasound source 17 is contained in an outer sleeve 24 that extends along the length of the shaft 12. The outer sleeve 24 is made from a biocompatible material such as Teflon or silicone. The outer sleeve 24 is attached to the handle 10 at a collar 25. The outer sleeve is preferably detachable from the handle 10, and is most preferably disposable. The probe unit also comprises a reservoir 122. The reservoir 122 may be located in the distal end of the probe unit 6, as shown in the embodiment of FIG. 2, or at the proximal end of the probe unit 6. The probe unit 6 is also provided with a delivery system which releases a substance 124 stored in the reservoir 122 from the distal end of the probe 6. The substance may be a pharmaceutical composition of the invention or an acoustic coupling medium for acoustic coupling of the ultrasound sonication to the body tissues, as explained below. Depressing a spring-biased push button 28 drives a piston 29 to create an elevated pressure in the reservoir 122 via a conduit 26 that urges the substance 124 to flow from the reservoir 122 out of the distal end 15 of the shaft 12.

The system 2 is also provided with a vacuum system that draws into the probe 6 substances surrounding the distal end of the shaft 12. A vacuum pump 28 may be located in the control unit 4, as shown in FIG. 1, or may be external to the control unit 4. The vacuum pump 28 creates a negative pressure in a receptacle 38 in the sleeve 24 via a vacuum hose 32 in the harness 8, and a connecting channel 34 in the handle 10. A normally closed valve 36 in the connecting channel 34 is opened by depressing a spring biased push-button switch 37 when it is desired to create a negative pressure in the receptacle 38, as explained below.

In an alternative embodiment (not shown) drawing of substances released from the gestational sac into the probe unit 6 utilizes a solution of high osmotic pressure that is applied to the external surface of the gestational sac. The high osmotic pressure solution draws amniotic and/or ceolomic fluid and dissolved or suspended substances across the fetal membranes by osmosis from the interior to the exterior of the gestational sac where the substances are collected in a receptacle.

The control unit contains a power supply 40 that is connected to the ultrasound transducer 17 via wires 44 in the harness 8 that connect with wires 45 in the probe unit 6. Closing a switch 41 on the handle 10 activates the ultrasound source 17 to the power supply 40. The control unit also contains a user input device, such as a key pad 42 that allows a user to input values of various parameters relating to the ultrasound sonication, such as intensity, pulse duration, pulse repetition rate or wavelength, as well as details relating to the individual being examined or the treatment.

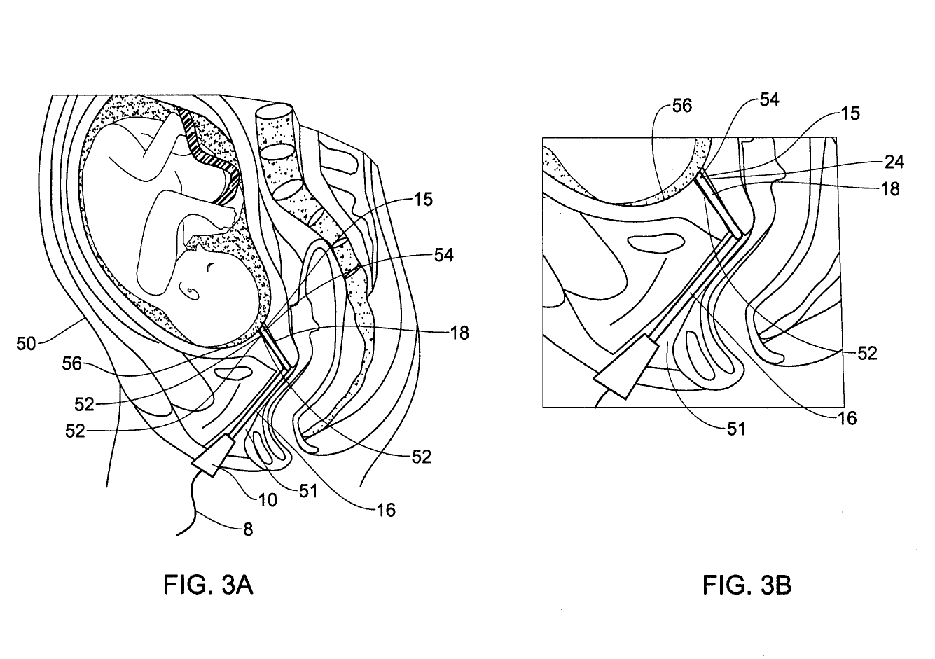

FIGS. 3a and 3b show use of the system 2 to collect a body substance such as an amniotic fluid sample or a coelomic fluid sample from an individual 50. The shaft 12 of the probe unit 6 is introduced into the vagina 51 and positioned with the vaginal portion 16 in the vagina 51 and the cervical portion 18 in the cervix 52. Positioning of the probe unit 6 in the body may be monitored by external ultrasonography to ensure proper placement of the probe unit 6 in the body. A small amount of coupling medium 24 is then expelled from the distal end 15 of the probe unit 6 by depressing the push-button 28. The distal end 15 of the probe is then apposed to a portion of the fetal membranes 56 adjacent to the cervix 52 in order to ensure acoustic coupling of ultrasound sonication to the portion 56 of the fetal membranes. Ultrasound sonication 54 emitted from the ultrasound source 17 is directed to the portion of the fetal membranes 56 adjacent to the cervix 52. The ultrasound activation button 41 is then depressed to activate the ultrasound transducer 17. Substances withdrawn from the gestational sac may be collected at any time by depressing the push-button 29 to open the vacuum valve 36. As demonstrated above, exposure of the fetal membranes 56 to the pharmaceutical composition of the invention increases the permeability of the fetal membranes. The permeability of the fetal membranes 56 may be monitored at any time by measuring the conductivity of the membranes (not shown). Substances passing out of the gestational sac as a result of the increased permeability, such as amniotic or coelomic fluid, are drawn into the distal end 15 of the probe unit 6 under the influence of the vacuum system and/or osmotic pressure when present, and are collected in the receptacle 38. After collection of substances passing through the fetal membranes, the vacuum is turned off, and the probe unit 6 is removed from the body. Substances collected in the receptacle 38 are then removed from the receptacle 16 and are analyzed.

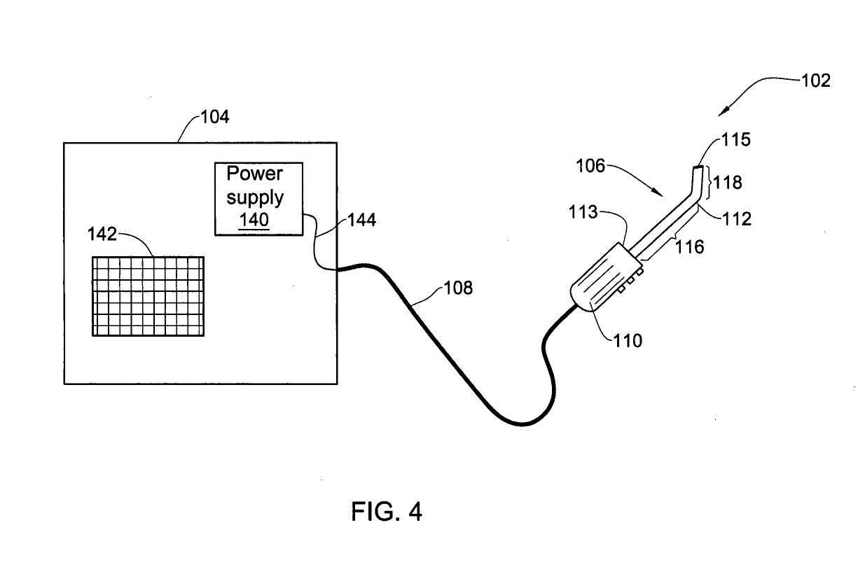

FIG. 4 shows a system generally indicated by 102 for transfetal membranes transport, in accordance with another embodiment of the invention. The system 102 may be used to transport substances such as drugs, from the exterior of the fetal membranes into the gestational sac.

The system 102 comprises a control unit 104 and a probe unit 106 which is attached to the control unit 104 via a harness 108. The probe unit 106 has a handle 110, a shaft 112, a proximal end 113 and a distal end 115. The shaft 112 may be rigid and permanently bent, or may be bendable to form a vaginal portion 116 and a cervical portion 118.

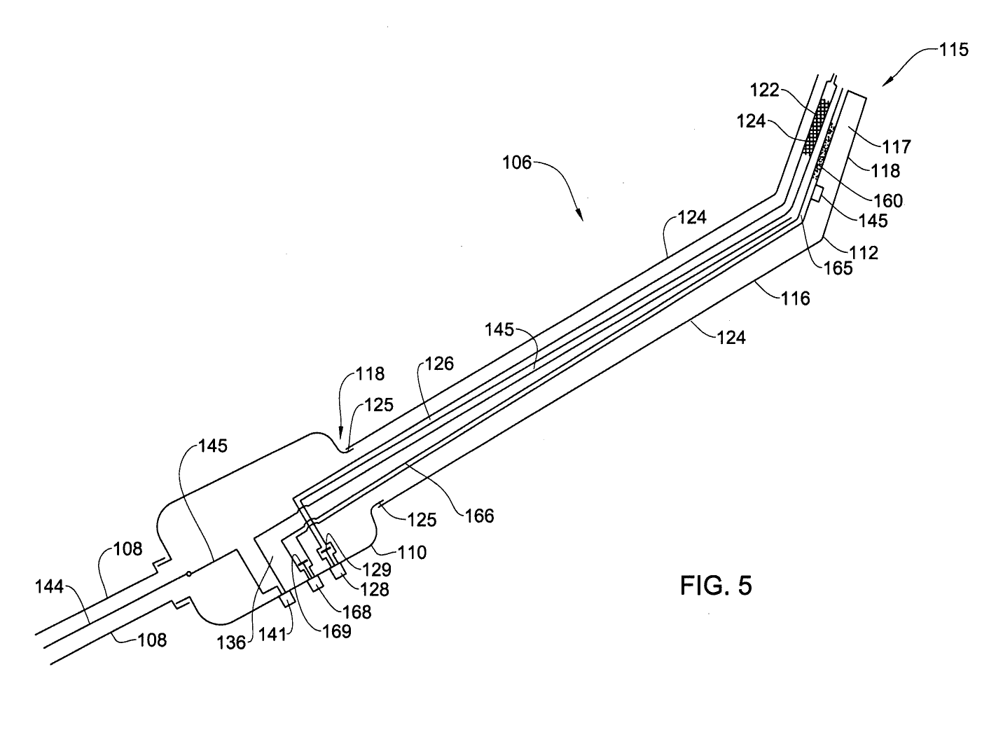

FIG. 5 shows the probe unit 106 in greater detail. The cervical portion 118 comprises an ultrasound source 117 that emits ultrasound waves from a distal end 115 of the probe unit 106. The ultrasound source 117 is contained in an outer sleeve 124 that extends along the length of the shaft 112. The outer sleeve 124 is attached to the handle 110 at a collar 125. The outer sleeve is preferably detachable from the handle 110, and is most preferably disposable. The probe unit 106 is also provided with a coupling medium delivery system which delivers an acoustic coupling medium to the distal end of the probe unit 106 for acoustic coupling of the ultrasound sonication to the body tissues, as explained below. A reservoir 122 is used to store an amount of an ultrasound coupling medium and/or a pharmaceutical composition of the invention 124. Depressing a spring-biased push button 128 drives a piston 129 to create an elevated pressure in the reservoir 122 via a conduit 126 that urges the coupling medium and/or pharmaceutical composition of the invention 124 to flow out from the reservoir 122 though the delivery tube 126 to the distal end 115 of the probe unit 112.

The system 102 is provided with a delivery system for delivering one or more substances, such as drugs and a pharmaceutical composition of the invention 124, to the external surface of the gestational sac. The drugs 160 are stored in a reservoir 165. Depressing a spring-biased push button 168 drives a piston 169 to create an elevated pressure in the reservoir 165 via a conduit 166 that urges the drugs to flow from the reservoir 165 out of the distal end 115 of the probe unit 112.

The control unit contains a power supply 140 that is connected to the ultrasound transducer 117 via wires 144 in the harness 108. Closing a switch 141 on the handle 110 activates the ultrasound source 117. The control unit also contains a user input device, such as a key pad 142 that allows a user to input values of various parameters relating to the ultrasound sonication, such as intensity, pulse duration, pulse repetition rate or wavelength, as well as details of the individual being examined.

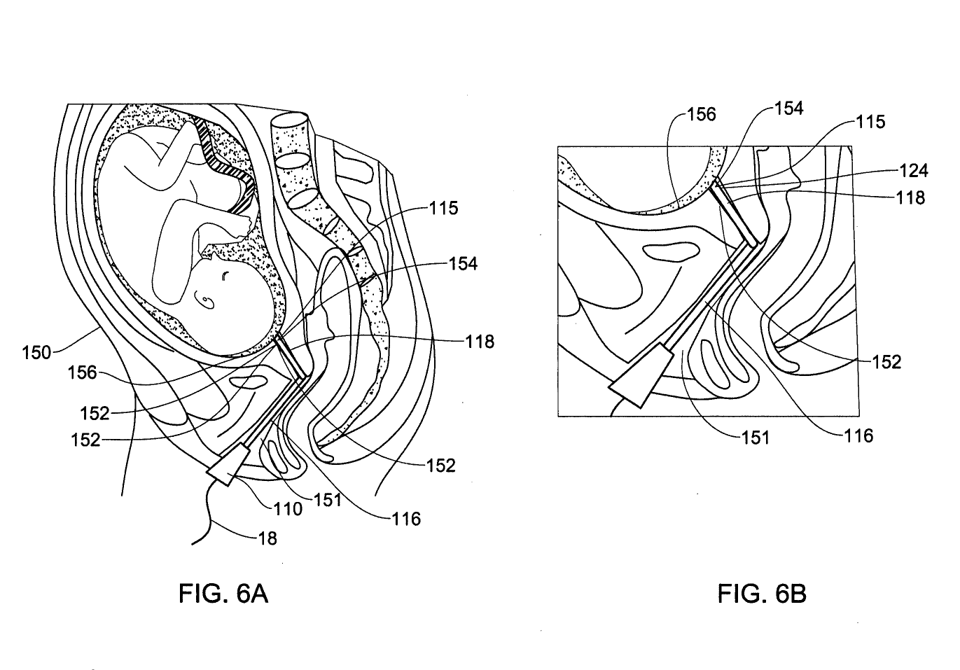

FIGS. 6a and 6b show use of the system 102 to deliver the one or more substances 160, such as a drug, into a gestational sac of an individual 150. The shaft 112 of the probe unit 106 is introduced into the vagina 152 and is positioned with the vaginal portion 116 in the vagina 151 and the cervical portion 118 in the cervix 152. A small amount of a pharmaceutical composition of the invention 124 is then delivered to the distal end 115 of the probe unit 106 by depressing the push-button 128. The distal end 115 of the probe is then apposed to a portion of the fetal membranes 156 adjacent to the cervix 152 in order to ensure acoustic coupling of ultrasound sonication to the portion 156 of the fetal membranes. The ultrasound activation button 141 is then depressed to activate the ultrasound source 117. The one or more substances 136 are delivered to the distal end 115 of the shaft 112 by depressing the push-button 168, during or after the ultrasound sonication. Ultrasound sonication 154 emitted from the ultrasound source 117 is directed to the portion of the fetal membranes 156 adjacent to the cervix 52. As demonstrated below, exposure of the fetal membranes 156 to the composition 124 the composition 124 with ultrasound sonication 154 increases the permeability of the fetal membranes. The permeability of the membranes 156 may be monitored during and after administration of the composition and sonication by measuring the electrical conductivity of the membranes (not shown). The substances 160 delivered to the distal end 115 of the shaft 112 are available to diffuse across the fetal membranes as a result of the increased permeability. After delivery of the substances 160, the probe unit 106 is removed from the body.

In one embodiment, the ultrasound sonication has a frequency of between about 20 kHz to about 3 MHz. In a preferred embodiment the ultrasound sonication has a frequency between about 20 kHz and about 500 kHz, and more preferably between about 20 kHz and 100 kHz. This range is referred to at times by the term “low frequency ultrasound sonication” (LFUS). In one embodiment, continuous ultrasound sonication for about 5 sec to about 30 min, more preferably, from about 30 sec to about 10 min, is used.

In another embodiment, the pharmaceutical composition of the invention may be applied prior to ultrasound sonication or after ultrasound sonication. The pharmaceutical composition may be applied for about 1 min. to about 30 min.

1.-23. (canceled)

24. A pharmaceutical composition for permeabilizing fetal membranes, comprising:
one or more active ingredients having a log K in the range of 2 to 4, K being the octanol/water partition coefficient; and
a physiologically acceptable carrier.

25. The pharmaceutical composition according to claim 24, wherein at least one of the one or more active ingredients is selected from the group consisting of bupivacaine, sodium lauryl sulfate (SLS), oleic acid, iso-stearic acid, lidocaine, ethylene glycol, cetyl trimethylammonium bromide (CTAB) and N-methy-2-pyrrolidone (NMP).

26. The pharmaceutical composition according to claim 24, wherein the one or more active ingredients are bupivacaine, SLS and/or limonene.

27. The pharmaceutical composition according to claim 24, being a paste or a liquid.

28. The pharmaceutical composition according to claim 24, wherein at least one of the one or more active ingredients is bupivacaine present in a concentration from 0.1% to 1% (wt/vol).

29. The pharmaceutical composition according to claim 24, wherein at least one of the one or more active ingredients is SLS present in a concentration from 0.1% to 10%.

30. The pharmaceutical composition according to claim 24, wherein at least one of the one or more active ingredients is oleic acid present in a concentration from 0.1% to 2%.

31. A system for transfetal membrane transport, comprising:
a probe unit adapted for insertion through a vagina to a into a female reproductive tract, the probe unit comprising a shaft having a proximal end and a distal end;
an ultrasound source located at the distal end of the shaft;
a reservoir;
a delivery system configured to release a substance in the reservoir from the distal end of the shaft; and
a control unit configured to activate the ultrasound source.

32. The system according to claim 31, wherein the shaft is curved or bent to form a vaginal portion and a cervical portion.

33. The system according to claim 31, wherein the shaft is flexible.

34. The system according to claim 31, further comprising a collecting system configured to collect substances around the distal end of the shaft.

35. The system according to claim 34, wherein the collecting system comprises a vacuum system.

36. The system according to claim 33, wherein the collecting system comprises a solution of high osmotic pressure.

37. The system according to claim 31, wherein the ultrasound source is configured to release ultrasound radiation having a frequency in the range of 20 kHz to 100 kHz.

38. A method for permeabilizing a fetal membrane, comprising:
applying to the fetal membrane the pharmaceutical composition according to claim 24.

39. The method according to claim 38, wherein the applying comprises releasing the pharmaceutical composition onto the fetal membrane from a system that comprises
a probe unit adapted for insertion through a vagina to a into a female reproductive tract, the probe unit comprising a shaft having a proximal end and a distal end,
an ultrasound source located at the distal end of the shaft,
a reservoir,
a delivery system configured to release a substance in the reservoir from the distal end of the shaft, and
a control unit configured to activate the ultrasound source.
the distal end of the shaft,
the releasing occurring at the distal end of the shaft.

40. The method according to claim 39, further comprising exposing the fetal membrane to ultrasound radiation.

41. A method for delivering one or more substances into an amniotic sac, comprising:
permeabilizing fetal membranes of the amniotic sac by the method of claim 38; and
applying the one or more substances to the permeabilized membrane.

42. The method according to claim 41, wherein the applying comprises releasing the one or more substances onto the fetal membrane from a system that comprises
a probe unit adapted for insertion through a vagina to a into a female reproductive tract, the probe unit comprising a shaft having a proximal end and a distal end,
an ultrasound source located at the distal end of the shaft,
a reservoir,
a delivery system configured to release a substance in the reservoir from the distal end of the shaft, and
a control unit configured to activate the ultrasound source.
the distal end of the shaft,
the releasing occurring at the distal end of the shaft.

43. A method for collecting a fluid from an amniotic sac, comprising:
permeabilizing fetal membranes of the amniotic sac according to the method of claim 38; and
collecting fluid released from the amniotic sac.

44. The method according to claim 43, wherein the fluid released from the amniotic sac is collected by a system that comprises
a probe unit adapted for insertion through a vagina to a into a female reproductive tract, the probe unit comprising a shaft having a proximal end and a distal end,
an ultrasound source located at the distal end of the shaft,
a reservoir,
a delivery system configured to release a substance in the reservoir from the distal end of the shaft, and
a control unit configured to activate the ultrasound source.
the distal end of the shaft,
the collecting occurring at the reservoir.

45. The method according to claim 43, further comprising applying ultrasound radiation to the fetal membrane.

46. The method according to claim 45, wherein the ultrasound radiation has a frequency in the range of from 20 kHz to 100 kHz.

47. A composition, comprising:
one or more active ingredients having a log K in the range of 2 to 4, K being the octanol/water partition coefficient; and
a physiologically acceptable carrier.
